(19) United States
(12) Patent Application Publication
(10) Pub. No.: US 2014/0243710 A1
(43) Pub. Date: Aug. 28, 2014

(54) POSTURE TRAINING SYSTEM AND CONTROL METHOD THEREOF

(71) Applicant: Electronics and Telecommunicatios Research Institute, Daejeon (KR)

(72) Inventor: Young Ho JEONG, Daejeon (KR)

(73) Assignee: Electronics and Telecommunicatios Research Institute, Daejeon (KR)

(21) Appl. No.: 14/038,369

(22) Filed: Sep. 26, 2013

(30) Foreign Application Priority Data
Feb. 27, 2013 (KR) 10-2013-0021601

(51) Int. Cl. A61B 5/11; A63B 24/00

(52) U.S. Cl. CPC A61B 5/1116; A63B 24/0003
USPC 600/594

(57) ABSTRACT

A posture training system and an operating method thereof are provided. The posture training system includes a sensor configured to sense information regarding user posture, a server configured to analyze the posture information obtained by the sensor and generate correction information for correcting the user posture, and a haptic unit configured to be wearable by a user and transmitting the correction information in a haptic manner to allow the user to correct his posture.

The present application claims the benefit of priority under 35 U.S.C. §119 to Korean Patent Application No. 10-2013-0021601 filed on Feb. 27, 2013, which is hereby incorporated by reference in its entirety.

### BACKGROUND OF THE INVENTION

1. Field of the Invention

The present invention relates to a posture training system and an operating method thereof, and more particularly, to a posture training system employing a haptic technology, and a control method (or an operating method) thereof.

2. Related Art

Haptic generally refers to two forces of tactile feedback felt when a skin comes into contact with a surface of an object and kinesthetic force felt when a motion of a joint and muscles are interfered. A haptic technology was commonly utilized mainly in a robotics field in 1970s to 1980s, and has extended to computer graphics and a virtual reality field since 1990s in its utilization field.

Haptic rendering refers to all the sequential calculation processes required for a user, who holds or wears a haptic device to recognize and manipulate a virtual object or the like, to feel realistic force between the virtual object and a physical device, and in this case, haptic rendering calculates and generates force according to properties and a state of an object and interaction between the object and a person. Here, the haptic device includes a force feedback device transferring physical force to muscles or a joint and a tactile feedback device transferring skin stimulus such as temperature, pressure, texture, vibration, pain, and the like, through a mechanical receptor existing in the skin. Among them, the force feedback device is mainly used for virtual reality, remote control, game, simulation, and the like. Namely, the force feedback device is used to recognize geometrical characteristics of an object of an object by providing kinaesthetia stimulus, and the tactile feedback device largely expresses texture by using an actuator such as a vibration motor, a pin arrangement, or the like, or feeds back a particular skin sensation.

The haptic technology has been applied to various fields, but it has so far limited to expression of haptic-related information by simply interworking with a virtual world or video contents, failing to meet consumers' demand with respect to novel haptic service experience. Thus, a method for providing a novel haptic-related service in conjunction with various user interface (UI) techniques such as gesture, and the like, overcoming limitations of existing haptic-related services, is required.

### SUMMARY OF THE INVENTION

The present invention provides a posture training system providing a haptic-related service to allow a user to conduct posture correction training, while receiving feedback regarding posture correction, and an operating method thereof.

In an aspect, a posture training system includes: a sensor configured to sense information regarding user posture; a server configured to analyze the posture information obtained by the sensor and generate correction information for correcting the user posture; and a haptic unit configured to be wearable by a user and transmitting the correction information in a haptic manner to allow the user to correct his posture.

The posture training system may further include: a connected device configured to transmit and receive information to and from the sensor, the haptic unit, and the server, transmit the sensed posture information from the sensor to the server, and transmit the generated correction information from the server to the haptic unit.

The sensor and the haptic unit may be separately provided or may be integrally provided in the user- wearable haptic device. The haptic device may be configured by using a user-wearable form, e.g., at least any one of a suit, vest, glove, joint protecting belt, and shoes. A plurality of sensors are provided in the haptic device, and a position sensor for sensing location information and a pressure sensor for sensing pressure information may be provided.

The server may include a haptic device database (DB) storing data regarding the haptic device and a posture model DB storing data regarding a posture model.

The server may further include: a posture analysis and correction information generating unit configured to compare the posture information sensed by the sensor and the stored posture model to generate the correction information; and a correction information converting unit configured to generate driving information of the haptic device corresponding to the correction information.

The connected device may be configured as a display device that may be able to play (or reproducing) a video, and play a video selected from the posture training video DB of the server.

In another aspect, a method for operating a posture training system, includes: sensing information regarding user's posture by using a sensor; comparing a posture model previously stored in a server with the posture information sensed by the server to generate correction information; converting the correction information into driving information of the haptic device user wears; and driving the haptic device on the basis of the driving information in order to correct the user's posture.

The posture information sensed by the sensor may be transmitted to the server through a connected device connected to the server via a network, and the driving information of the haptic device may be transmitted to the haptic device through the connected device.

The server may include a haptic device database (DB) storing data regarding the haptic device, and the method may further include: sensing a type of the haptic device the user wears on the basis of the information transmitted from the haptic device, wherein the generating of the correction information and the converting the correction information into the driving information of the haptic device may be performed on the basis of the type of the haptic device sensed in the operation of sensing a type of the haptic device.

According to embodiments of the present invention, an error based on subjective judgment of a trainer and semantic ambiguity according to verbal expression delivered by the trainer, that may be generated during a posture training process in various items of sports such as golf, dance, yoga, archery, and the like, are eliminated, so a learner can conduct accurate and intuitive posture training for himself or herself, without help of a trainer, and thus, training cost can be reduced and training effects can be maximized.

### BRIEF DESCRIPTION OF THE DRAWINGS

FIG. 1 is a schematic view illustrating a configuration of a posture training system according to an embodiment of the present invention;

FIG. 2 is a perspective view illustrating installation positions of sensors of a haptic device in FIG. 1;

FIG. 3 is a block diagram illustrating a configuration of a server in FIG. 1; and

FIG. 4 is a flow chart illustrating an operating method of the posture training system of FIG. 1.

### DESCRIPTION OF EXEMPLARY EMBODIMENTS

Hereinafter, a posture training system and an operating method thereof according to embodiments of the present invention will be described in detail with reference to the accompanying drawings. In the following description, position relationships between and among components are basically based on the drawings. In the drawings, the structure of the invention may be simplified for the description purpose or may be exaggerated as necessary. Thus, the present invention is not limited thereto and may be implemented by adding, modifying or omitting various devices.

Also, in the present embodiment, a posture training system for exercising golf swing and an operating method thereof will be described, but it is merely an example and the present invention is not limited thereto and may be applied to a posture training system of various items of sports such as yoga, archery, and dance requiring accurate posture or various fields, such as a medical field, a game field, a defense industry field, an aerospace field, and the like, in which posture training is required for a specific purpose.

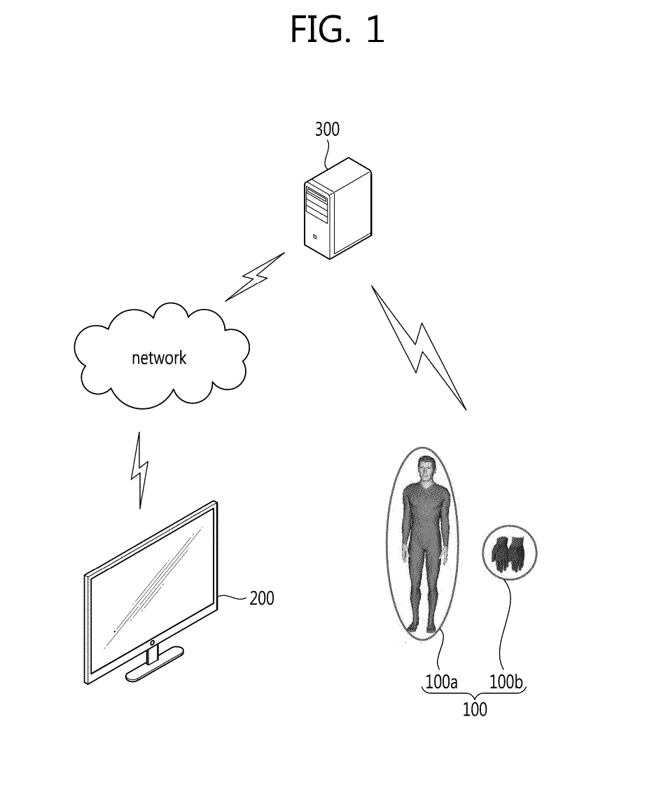

FIG. 1 is a schematic view illustrating a configuration of a posture training system according to an embodiment of the present invention.

As illustrated in FIG. 1, a posture training system according to the present embodiment may include a haptic device 100 that a user wears, a server 300 storing and calculating various types of information, and a connected device 200 connecting the haptic device 100 and the server 300.

Here, the haptic device 100 is formed to allow a user to wear it, and includes a plurality of sensors (not shown) that may detect information regarding a user's posture, and a haptic unit (not shown) driven to correct a user's posture.

As illustrated in FIG. 1, the haptic device 100 is configured as a wearable device. In the present embodiment, the haptic device 100 is configured to include a whole-body suit 100a and a glove 100b. However, it is merely an example and the haptic device 100 may be configured by using various clothes structure such as a top suit, a bottom suit, a whole-body suit, a vest, a glove, a joint protecting belt, a band, shoes, and the like.

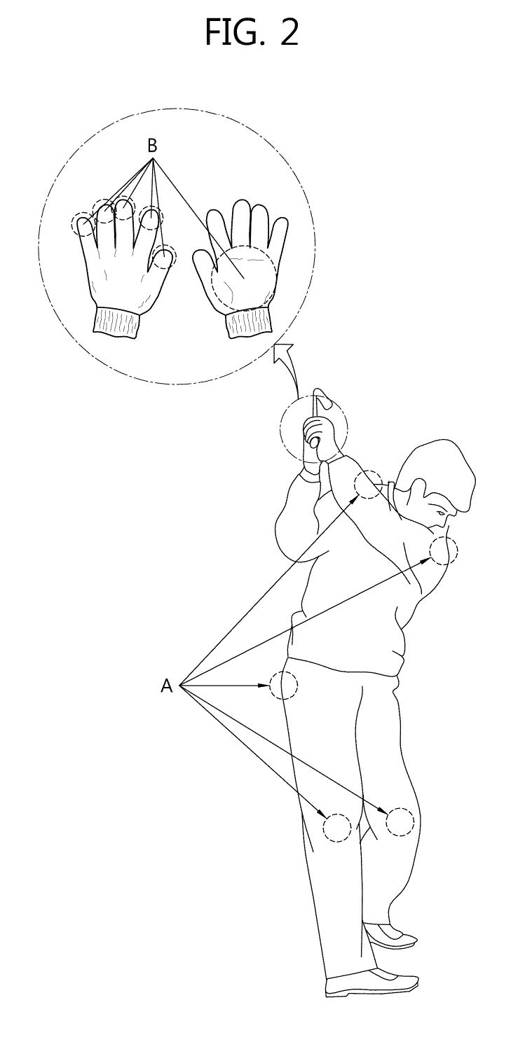

FIG. 2 is a perspective view illustrating installation positions of sensors of the haptic device in FIG. 1. A plurality of sensors are provided in major positions of the haptic device 100. In particular, as illustrated in FIG. 2, the plurality of sensors may be configured to be positioned in major joint parts such as the shoulder, the elbow, the wrist, the waist, the knee, the ankle, and the like (please See ‘A’ in FIG. 2). The sensors are configured as position sensors and sense positions when a user takes a particular pose or motion, thus sensing information regarding a user's posture. Thus, as illustrated in FIG. 2, when the user, wearing the haptic device 100, conducts a swing training, a posture changing process according to a user's swing trace may be sensed as three-dimensional (3D) information.

Also, the plurality of sensors may include a pressure sensor, as well as a position sensor. In a golf swing training, the way in which the user grips a golf club, as well as a body posture, may be an important factor. Thus, as illustrated in FIG. 2, in the present embodiment, a plurality of pressure sensors are provided in portions of the globe of the haptic device 100, whereby a user's grip method in practicing his swing may be sensed (please see B in FIG. 2).

In this manner, since a plurality of sensors including a position sensor and a pressure sensor are installed in positions of a globe, shoes, and the like, whereby the sensors can be applied to be used to monitor various types of information such as the way in which the user grips an implement while training a posture or the way in which the center of gravity is shifted while training, and the like. In addition, besides the position sensor and the pressure sensor, various other sensors may be configured to sense various types of posture information according to the purpose of posture training and content of posture.

However, in the present embodiment, the sensors sensing user posture information are integrally installed in the haptic device 100, but this is merely an example and a wearable sensing device, apart from the haptic device 100, may also be configured and user posture information may be sensed by using sensors installed in a training space or in a connected device.

Meanwhile, the haptic unit of the haptic device 100 is configured to provide correction information to the user during the posture training. The haptic unit may be able to transmit correction information to the user in a manner of transmitting physical force or providing tactile sensation such as pressure, vibration, temperature, and the like, in a state in which the haptic unit is in contact with the user body. The haptic device may be configured by using various members such as a vibration motor, a pin actuator, and the like. Since various conventional members are utilized, a detailed description thereof will be omitted.

As described above, the haptic device 100 may sense various types of posture information by using sensors while the user is in a posture training, and drive the haptic unit to provide posture correction information to the user, whereby the user can perform posture training while correcting his posture by himself.

Meanwhile, the connected device 200 is configured to be connected to the haptic device 100 through a fixed line or wirelessly to transmit and receive information, and also wirelessly connected to the server 300 to transmit and receive information. The connected device 200 receives sensed posture information from the sensors of the haptic device 100 and transmits the same to the server 300 and transmits haptic driving information, or the like, transferred from the server 300 to the haptic device 100, serving as a medium for transmitting various types of information between the haptic unit and the server 300.

In detail, in the present embodiment, the connected device 200 and the haptic device 100 may be connected in a near field communication (NFC) manner, and the connected device 200 and the server may be connected via a network such as the Internet. However, this is merely an example, and various types of information may be transmitted therebetween by using various signal transmitting and receiving scheme.

The connected device 200 may be configured by using various devices, and in the present embodiment, it may be configured by using a display device such as a smart TV. The display device is connected to the server 300 via the Internet and may also be connected to the haptic device 100 in the NFC manner. Also, the display device may execute an agent for posture training and display the same on a display, whereby the user may set training content and undergo training by using the displayed agent. In addition, the display device may play a posture training video stored in the server 300 and may be configured to display a user's posture on a screen on the basis of the user posture information sensed by the haptic device.

The server 300 stores various contents related to posture training and analyzes user posture information sensed by the haptic device 100 to generate correction information. Hereinafter, the configuration of the server according to the present embodiment will be described in detail.

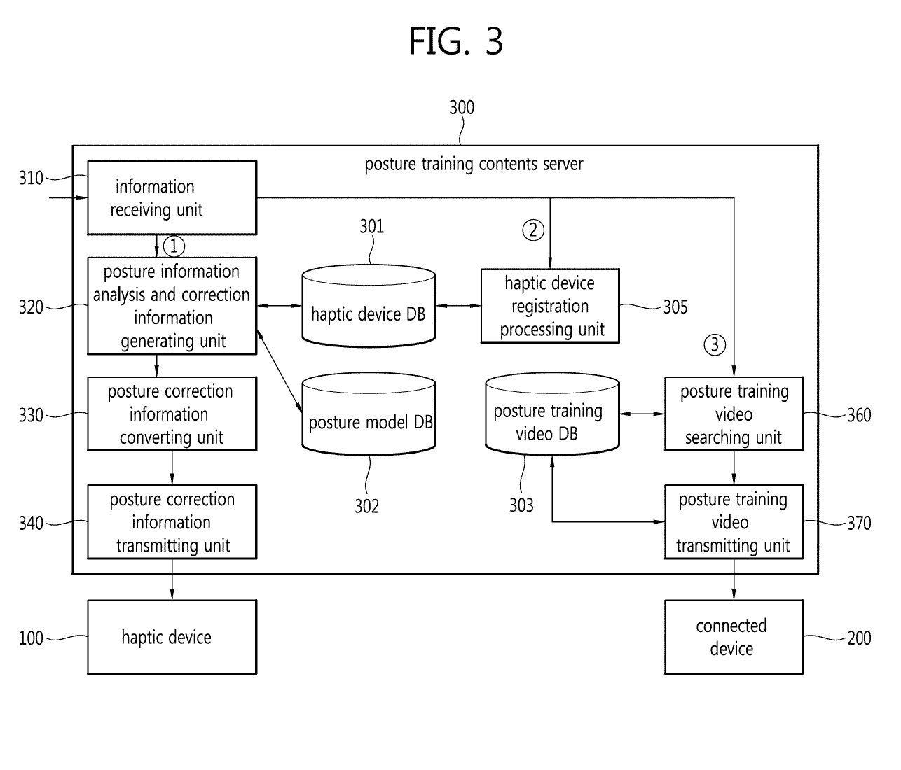

FIG. 3 is a block diagram illustrating a configuration of a server in FIG. 1. As illustrated in FIG. 3, the server 300 according to the present embodiment may include a haptic device database (DB) 301, a posture model DB 302, and a posture training video DB 303.

The haptic device DB 301 may store various types of information regarding various types of haptic devices. The haptic device 100 may be configured as various types according to events of posture training and content of posture training. Thus, on the basis of ID information transmitted from the haptic device 100 the user wears, a type of the haptic device 100 being currently used may be determined and user posture information may be analyzed in consideration of the determined type of the haptic device 100.

The posture model DB 302 may store various types of posture model information as textbook of posture training. The posture model DB 302 may store various types of data according to events of posture training and content of posture training, and store various posture models (e.g., a posture model of a sports athlete selected by a user) of the same posture training to allow a user to select a desired model.

The posture training video DB 303 may store various posture training videos that may be played (or reproduced) through the connected device 200 during posture training.

Meanwhile, the server 300 includes an information receiving unit 310 receiving various types of information from the outside. In the present embodiment, the information receiving unit 310 is configured to receive information transmitted through the connected device 200, and besides, it may also be configured to directly receive information from the haptic device 100 or may be configured to receive information from a component other than the connected device 200 and the haptic device 100.

Here, the received information may include ID information of the haptic device the user wears, user posture information transmitted from the haptic device during training, posture training video information selected by the user, and the like. The server 300 may process the received information through various routines according to types of the information.

In detail, in a case in which the received information is ID information of the haptic device 100, the haptic device registration processing unit 350 compares the received ID information of the haptic device with information stored in the haptic device DB 301 to determine a type of the haptic device the user wears (please see \hat{2} in FIG. 3). Thus, the type of the haptic device the user wears ca be recognized among various types of haptic devices such as shoot, glove, vest, and the like.

Also, when the received information is user posture information sensed by the sensors, a posture information analysis and correction information generating unit 320 may analyze posture the user posture information with reference to data corresponding to the ID of the haptic device 100 (please see \hat{1} in FIG. 3). The posture information analysis and correction information generating unit 320 may compare numerical values with posture model data stored in the posture model DB 302 and analyze the comparison results to generate correction information regarding required posture correction.

A posture correction information converting unit 330 of the server 300 may perform a process of converting the generated correction information into an appropriate haptic driving signal corresponding to the corresponding correction information. For example, when it is determined that the shoulder is excessively high in a golf address posture according to the posture analysis results, requiring posture correction to lower shoulder, a haptic driving signal for appropriately expressing information of lowering the shoulder may be generated through the haptic unit, of the haptic device 100 the user wears, provided in an upper portion of the shoulder. The driving signal may be variously formed according to a configuration of the haptic unit of the haptic device 100, and the user may recognize the correction information when the haptic device is driven. The driving of the haptic device 100 may be provided such that the user may recognize correction content by intuition, or the correction information may be transmitted by driving the haptic device according to an agreed method the user may be able to recognize through a manual orientation.

In this manner, haptic driving information converted by the posture correction information converting unit 330 may be transmitted to the haptic device 100 through a posture correction information transmitting unit 340. In FIG. 3, it is illustrated that the converted haptic driving information is directly transmitted from the server 300 to the haptic device 100, but it may mean a final transmission destination, which may have omitted a process of going through the connected device 200 midway.

Meanwhile, when the received information is video information selected by the user, a posture training video searching unit 360 searches and selects the video selected by the user from the posture training video DB 303. A posture training video transmitting unit 370 is configured to transmit video information that may be played in the connected device 200.

In this manner, the server 300 of the posture training system according to the present embodiment may store various contents related to the posture training, and when information is received from the outside, the server 300 may process the received information by using the stored data and a calculation structure according to a type of the received information. In the present embodiment, the scheme in which the server processes three types of information has been described, but besides, given information may be process in various manners such that a three-dimensional (3D) video is generated from the user posture information, correction information is displayed on the connected device, and the like.

As described above, the posture training system according to the present embodiment is configured to obtain a user's posture as 3D information from the haptic device 100 the user wears, analyze the obtained 3D information, and allow the user to recognize derived correction information in a haptic manner during training. Thus, an error based on subjective judgment of a trainer and semantic ambiguity according to verbal expression delivered by the trainer, that may be generated during a posture training process as in the related art can be eliminated, so a learner can conduct accurate and intuitive posture training for himself or herself.

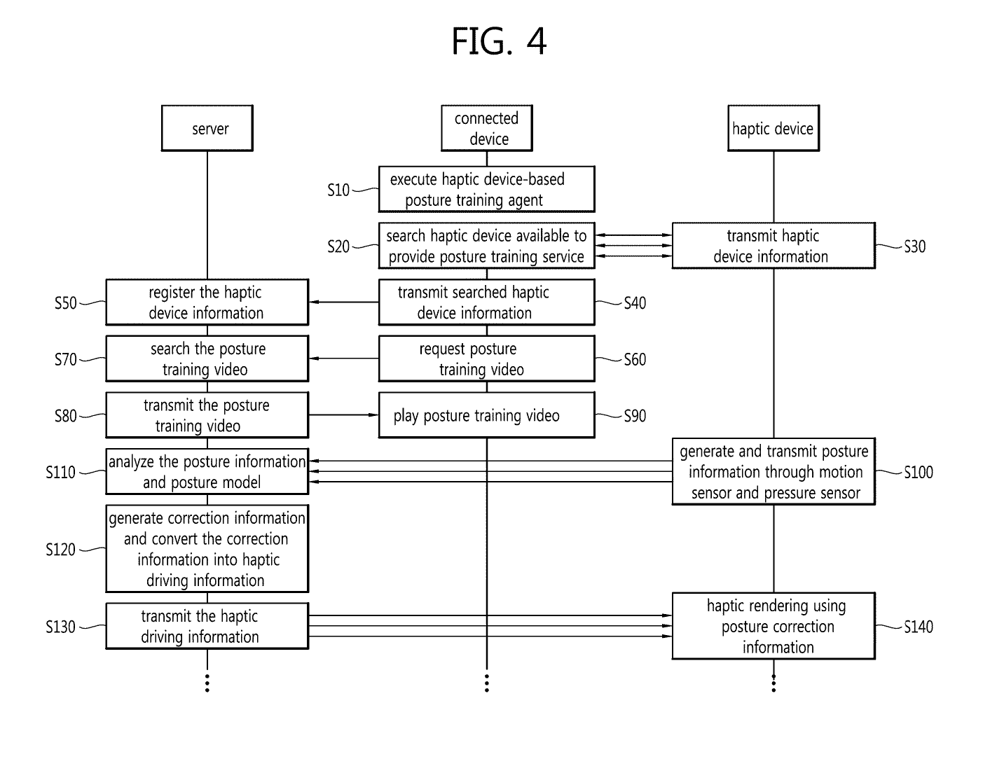

FIG. 4 is a flow chart illustrating an operating method of the posture training system of FIG. 1. Hereinafter, a specific operating method of the posture training system according to the present embodiment will be described with reference to FIG. 4.

First, an agent for posture training is executed by using a smart TV corresponding to the connected device 200 (S10). Through the execution of the agent, the connected device 200 is maintained to be connected to the server 300 through the Internet.

When the agent is executed, the connected device 200 searches the haptic device maintained in an ON state in an adjacent location (S20). When a search signal from the connected device 200 is sensed, the haptic device 100 the user wears transmits its ID information to the connected device 200 (S30). Communication between the connected device 200 and the haptic device 100 may be automatically performed through NFC. However, besides, information regarding the haptic device 100 the user wears may be directly selected from an agent menu of the connected device 200.

Meanwhile, when the ID information of the haptic device 100 the user wears is sensed, the connected device 200 transmits the information to the server 300 (S40). The haptic device registration processing unit 350 of the server 300 searches the haptic device DB 301 for the received ID information of the haptic device 100 to recognize the haptic device 100 being currently used (S50) and operates the training system in consideration thereof.

The user requests a training video desired to be referred during training through a display of the connected device 200 (S60), and the posture training video searching unit 360 of the server 300 searches the posture training video DB 303 for the requested training video (S70). The searched video is transmitted to the connected device 200 (S80), and the connected device 200 plays the corresponding video (S90) so the user can obtain (or learn) basic information required for posture training with reference to the video.

Meanwhile, when the user starts to conduct posture training with reference to the video, the haptic device 100 senses posture information by using the plurality of sensors installed therein and transmits the sensed posture information to the server 300 in real time through the connected device 200 (S100). The posture information analysis and correction information generating unit 320 of the server 300 analyzes the posture model stored in the posture model DB 302 and the received posture information to generate correction information (S110). Thereafter, the posture correction information converting unit 330 of the server 300 converts the generated correction information into corresponding haptic driving information (S120), and the haptic driving information is transmitted to the haptic device 100 through the posture correction information transmitting unit 340 (S130). The haptic device 100 is driven according to the corresponding information to provide stimulus in a haptic manner to the user body, and thus, the user can correct his or her posture by intuition (S140).

As described above, the method for operating the posture training system according to embodiments of the present invention has been described based on an example of typical operations, and some of the operations may modified or additional operations may be performed. For example, according to the operating method as described above, posture information sensed by sensors of the haptic device are all transmitted to the server to generate correction information, but in case of a haptic device such as a haptic glove, or the like, pressurization force of the user's hand may be sensed by the pressure sensor installed therein, and when the sensed pressurization force exceeds a pre-set pressure level, the haptic unit may be driven, whereby the haptic device may generate correction information by itself. Also, in another example, in the operating method as described above, only correction information is transmitted to the user by driving the haptic device, but correction information may be display through the display of the connected device so the user may recognize the correction information visually, as well as in a haptic manner.

While the present invention has been shown and described in connection with the embodiments, it will be apparent to those skilled in the art that modifications and variations can be made without departing from the spirit and scope of the invention as defined by the appended claims.

What is claimed is:

1. A posture training system comprising:
a sensor configured to sense information regarding user posture;
a server configured to analyze the posture information obtained by the sensor and generate correction information for correcting the user posture; and
a haptic unit configured to be wearable by a user and transmitting the correction information in a haptic manner to allow the user to correct his posture.

2. The posture training system of claim 1, further comprising:
a connected device configured to transmit and receive information to and from the sensor, the haptic unit, and the server, transmit the sensed posture information from the sensor to the server, and transmit the generated correction information from the server to the haptic unit.

3. The posture training system of claim 2, wherein the sensor and the haptic unit are integrally provided in the user- wearable haptic device.

4. The posture training system of claim 3, wherein the haptic device is configured to include at least any one of a suit, vest, glove, joint protecting belt, and shoes the user is wearable.

5. The posture training system of claim 3, wherein a plurality of sensors are provided in the haptic device, and a position sensor for sensing location information and a pressure sensor for sensing pressure information are provided.

6. The posture training system of claim 3, wherein the server comprises a haptic device database (DB) storing data regarding the haptic device and a posture model DB storing data regarding a posture model.

7. The posture training system of claim 6, wherein the server further comprises a posture analysis and correction information generating unit configured to compare the posture information sensed by the sensor and the stored posture model to generate the correction information, and a correction information converting unit configured to generate driving information of the haptic device corresponding to the correction information.

8. The posture training system of claim 3, wherein the connected device is a display device able to reproduce an image.

9. The posture training system of claim 3, wherein the server further comprises a posture training video DB configured to store a posture training video, and the connected device is configured to play a video selected from the posture training video DB.

10. A method for operating a posture training system, the method comprising:
sensing information regarding user's posture by using a sensor;
comparing a posture model previously stored in a server with the posture information sensed by the server to generate correction information;
converting the correction information into driving information of the haptic device user wears; and
driving the haptic device on the basis of the driving information in order to correct the user's posture.

11. The method of claim 10, wherein the posture information sensed by the sensor is transmitted to the server through a connected device connected to the server via a network, and
the driving information of the haptic device is transmitted to the haptic device through the connected device.

12. The method of claim 11, wherein the sensor is integrally provided in the haptic device.

13. The method of claim 12, wherein a plurality of sensors are provided in the haptic device, and a position sensor for sensing location information and a pressure sensor for sensing pressure information are provided.

14. The method of claim 11, wherein the server comprises a haptic device database (DB) storing data regarding the haptic device, and
the method further comprising:
sensing a type of the haptic device the user wears on the basis of the information transmitted from the haptic device,
wherein the generating of the correction information and the converting the correction information into the driving information of the haptic device is performed on the basis of the type of the haptic device sensed in the operation of sensing a type of the haptic device.

15. The method of claim 11, wherein the server further comprises a posture analysis and correction information generating unit configured to compare the posture information sensed by the sensor and the stored posture model to generate the correction information, and a correction information converting unit configured to generate driving information of the haptic device corresponding to the correction information.

16. The method of claim 11, wherein the connected device is a display device able to reproduce an image.

17. The method of claim 16, wherein the server further comprises a posture training video DB configured to store a posture training video, and
the method further comprising:
playing the posture training video stored in the server through the connected device.

18. A posture training system comprising:
a haptic device configured to be wearable by a user and including a plurality of sensors configured to sense information regarding user's posture and a haptic unit configured to transmit correction information in a haptic manner to allow the user to correct his posture;
a server configured to analyze the posture information obtained by the sensor and generate correction information for the user to correct his posture; and
a connected device connected to the haptic device and the server, transmit information received from the haptic device to the server, and transmit information received from the server to the haptic device.

19. The posture training system of claim 18, wherein the connected device is configured as a display device able to play the posture training video stored in the server.

20. The posture training system of claim 19, wherein the server further comprises a posture analysis and correction information generating unit configured to compare the posture information sensed by the sensor and the stored posture model to generate the correction information, and a correction information converting unit configured to generate driving information of the haptic device corresponding to the correction information.
